SEQUENCE LISTING (6 sequences)

SEQ ID NO: 1 - protein, 583 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 2 - protein, 583 aa, Artificial Sequence
  SEQ ID NO 2 Exemplary CFI Amino Acid Sequence
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVAQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 3 - DNA, 1807 bp, Artificial Sequence
  SEQ ID NO 3- Exemplary Codon Optimized Human Complement Factor I
gcggccgccaccatgaaactgctgcatgtctttctgctgtttctgtgcttccatctgcgcttctgcaaggtcacttacacttctcaggaggatctggtcgagaagaagtgtctggccaagaagtacacacacctgagctgcgacaaggtgttctgtcagccttggcagagatgcatcgagggcacctgcgtgtgcaagctgccttaccagtgcccaaagaacggaaccgccgtgtgcgcaacaaatcggcggagctttccaacatattgccagcagaagagcctggagtgtctgcaccccggcaccaagttcctgaacaatggcacctgcacagccgagggcaagttttctgtgagcctgaagcacggcaacacagatagcgagggcatcgtggaggtgaagctggtggaccaggataagaccatgttcatctgtaagagctcctggtccatgagggaggcaaacgtggcatgcctggatctgggattccagcagggagcagacacacagaggcgctttaagctgtccgacctgtctatcaatagcaccgagtgcctgcacgtgcactgtaggggcctggagacatccctggcagagtgcaccttcacaaagcggagaaccatgggctaccaggactttgccgacgtggtgtgctatacccagaaggccgatagcccaatggacgatttctttcagtgcgtgaacggcaagtatatctcccagatgaaggcctgcgacggcatcaatgactgtggcgatcagtctgacgagctgtgctgtaaggcctgtcagggcaagggcttccactgcaagagcggcgtgtgcatcccttcccagtaccagtgcaacggcgaggtggattgtatcacaggagaggacgaagtgggatgcgctgccgccagacacccaaccatccagggctttgcctctgtggcccaggaggagacagagatcctgacagccgacatggatgccgagaggcgccggatcaagtctctgctgcccaagctgagctgcggcgtgaagaataggatgcacatcagaaggaagcgcatcgtgggaggcaagagggcacagctgggcgatctgccttggcaggtggccatcaaggacgcctctggcatcacctgcggcggcatctacatcggaggatgttggatcctgaccgcagcacactgcctgagagcaagcaagacacacaggtatcagatttggaccacagtggtggattggatccacccagacctgaagagaatcgtgatcgagtacgtggataggatcatcttccacgagaactacaatgccggcacatatcagaacgacatcgccctgatcgagatgaagaaggatggcaataagaaggactgtgagctgccacgctccatccctgcatgcgtgccctggagcccctatctgttccagcccaacgatacctgtatcgtgtccggctggggccgcgagaaggacaatgagcgggtgttttctctgcagtggggcgaggtgaagctgatctccaactgttctaagttctacggcaatcggttttatgagaaggagatggagtgcgccggcacctacgatggcagcatcgacgcctgtaagggcgattccggaggaccactggtgtgcatggacgcaaacaatgtgacatacgtgtggggcgtggtgtcctggggcgagaattgcggcaagccagagtttcccggcgtgtataccaaggtggccaactattttgattggatttcctaccatgtcgggagaccattcatttcacagtataacgtgtaataagcttggatccagatct

SEQ ID NO: 4 - DNA, 1807 bp, Artificial Sequence
  SEQ ID NO 4- Exemplary Non-Codon Optimized Human Complement Factor I
gcggccgccaccatgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcagcagctagacatcctacaattcaaggctttgcatctgtggctcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataataagcttggatccagatct

SEQ ID NO: 5 - DNA, 1752 bp, Artificial Sequence
  SEQ ID NO 5 Exemplary CFI Nucleotide Sequence
atgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtgactcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataa

SEQ ID NO: 6 - DNA, 1752 bp, Artificial Sequence
  SEQ ID NO 6- Exemplary CFI Nucleotide Sequence
atgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtggctcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataa

(19) United States
(12) Patent Application Publication
(10) Pub. No.: US 2022/0347265 A1
(43) Pub. Date: Nov. 3, 2022

(54) METHODS FOR TREATING PATIENTS HAVING CFI MUTATIONS WITH RECOMBINANT CFI PROTEINS

(71) Applicant: Gemini Therapeutics Sub, Inc., Cambridge, MA (US)

(72) Inventors: James McLaughlin, Cambridge, MA (US); Lisa Huang, Cambridge, MA (US); Scott Lauder, Andover, MA (US); Suresh Katti, Hamden, CT (US); Adam Tellier, Somerville, MA (US)

(21) Appl. No.: 17/771,330

(22) PCT Filed: Oct. 23, 2020
(86) PCT No.: PCT/US2020/057093

§ 371 Date: Apr. 22, 2022

Related U.S. Application Data
(60) Provisional application No. 62/925,066, filed on Oct. 23, 2019.

(51) Int. Cl. A61K 38/17

(52) U.S. Cl. CPC A61K 38/1725

(57) ABSTRACT

The present disclosure provides methods for treating, preventing, or inhibiting diseases in patients having one or more CFI mutations.

### CROSS-REFERENCE TO RELATED APPLICATIONS

This application claims the benefit of and priority to U.S. Provisional Patent Application No. 62/925,066, filed Oct. 23, 2019, the disclosures of which are incorporated by reference herein in their entireties for all purposes.

### SEQUENCE LISTING

The instant application contains a Sequence Listing which has been submitted electronically in ASCII format and is hereby incorporated by reference in its entirety. Said ASCII copy, created on Oct. 20, 2020, is named GEM-014WO_SL_ST25.txt and is 20,551 bytes in size.

### BACKGROUND OF THE DISCLOSURE

Age-related macular degeneration (AMD) is a medical condition and is the leading cause of legal blindness in Western societies. AMD typically affects older adults and results in a loss of central vision due to degenerative and neovascular changes to the macula, a pigmented region at the center of the retina which is responsible for visual acuity. There are four major AMD subtypes: Early AMD; Intermediate AMD; Advanced non-neovascular (“Dry”) AMD; and Advanced neovascular (“Wet”) AMD. Typically, AMD is identified by the focal hyperpigmentation of the retinal pigment epithelium (RPE) and accumulation of drusen deposits and/or geographic atrophy. The size and number of drusen deposits or level geographic atrophy typically correlates with AMD severity.

AMD occurs in up to 8% of individuals over the age of 60, and the prevalence of AMD continues to increase with age. The U.S. is anticipated to have nearly 22 million cases of AMD by the year 2050, while global cases of AMD are expected to be nearly 288 million by the year 2040.

There is a need for novel treatments for preventing progression from early to intermediate and/or from intermediate to advanced stages of AMD to prevent loss of vision, particularly in certain subpopulations of patients having one or more CFI mutations.

### SUMMARY OF THE DISCLOSURE

The disclosure provides compositions and methods for treating, preventing, or inhibiting a disease or disorder associated with one or more CFI mutations.

In one aspect, the disclosure provides for a method of treating a subject having a disease or disorder associated with undesired activity of the alternative complement pathway, comprising the step of administering to the subject a complement factor I (CFI) protein or biologically active fragment and/or variant thereof, wherein the subject has one or more CFI gene mutations. In some embodiments, the disclosure provides for a method of treating a subject having age-related macular degeneration (AMD), comprising the step of administering to the subject a complement factor I (CFI) protein or biologically active fragment and/or variant thereof, wherein the subject has one or more CFI gene mutations. In some embodiments, the CFI protein or biologically active fragment and/or variant thereof comprises a heavy chain and a light chain. In some embodiments, the CFI protein or biologically active fragment and/or variant thereof comprises a FIMAC domain. In some embodiments, the CFI protein or biologically active fragment and/or variant thereof comprises a Scavenger Receptor Cysteine Rich (SRCR) domain. In some embodiments, the CFI protein or biologically active fragment and/or variant thereof comprises at least one (low-density lipoprotein) LDL receptor Class A domain. In some embodiments, the CFI protein or biologically active fragment and/or variant thereof comprises two LDL receptor Class A domains. In some embodiments, the CFI protein or biologically active fragment and/or variant thereof comprises a serine protease domain. In some embodiments, the CFI protein or biologically active fragment and/or variant thereof comprises a FIMAC domain, a Scavenger Receptor Cysteine Rich (SRCR) domain, and two LDL receptor Class A domains. In some embodiments, the CFI protein or biologically active fragment and/or variant thereof is capable of cleaving C3b and C4b proteins. In some embodiments, the CFI protein or biologically active fragment and/or variant thereof is capable of inhibiting the assembly of C3 and C5 convertase enzymes. In some embodiments, the CFI protein or biologically active fragment and/or variant thereof comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 1, or a biologically active fragment thereof. In some embodiments, the CFI protein or biologically active fragment and/or variant thereof comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 2, or a biologically active fragment thereof. In some embodiments, the CFI protein or biologically active fragment and/or variant thereof is encoded by a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID NOs: 3-6 or a fragment thereof (e.g., a fragment encoding a biologically active fragment of a CFI protein). In some embodiments, the CFI protein or biologically active fragment and/or variant thereof is a mature CFI protein or biologically active fragment and/or variant thereof. In some embodiments, the subject is not administered a protease or a polynucleotide encoding a protease (e.g., not administered a protease or a polynucleotide encoding a protease before, after, or concurrently with the CFI protein or biologically active fragment and/or variant thereof). In some embodiments, the subject is not administered a furin protease or a polynucleotide encoding a furin protease (e.g., not administered a protease or a polynucleotide encoding a protease before, after, or concurrently with the CFI protein or biologically active fragment and/or variant thereof). In some embodiments, the subject is a human. In some embodiments, the human is at least 40 years of age. In some embodiments, the human is at least 50 years of age. In some embodiments, the human is at least 65 years of age. In some embodiments, the CFI protein or biologically active fragment and/or variant thereof is administered locally. In some embodiments, the CFI protein or biologically active fragment and/or variant thereof is administered systemically. In some embodiments, the subject has a loss-of-function mutation in the subject's CFI gene. In some embodiments, the subject has one or more CFI mutations selected from the group consisting of: G119R, L131R, V152M, G162D, R187Y, R187T, T2031, A240G, A258T, G287R, A300T, R317W, R339Q, V412M, and P553S. In some embodiments, the subject has one or more CFI mutations selected from the group consisting of: P553S, K441R, R339Q, R339Ter, R317Q, R317W, A300T, G287R, G261D, A258T, A240G, T2031, R187Q, R187Ter, G162D, V152M, and G119R. In some embodiments, the subject has a P553S CFI mutation. In some embodiments, the subject has a K441R CFI mutation. In some embodiments, the subject has an R339Q CFI mutation. In some embodiments, the subject has an R339Ter CFI mutation. In some embodiments, the subject has an R317Q CFI mutation. In some embodiments, the subject has an R317W CFI mutation. In some embodiments, the subject has an A300T CFI mutation. In some embodiments, the subject has a G287R CFI mutation. In some embodiments, the subject has a G261D CFI mutation. In some embodiments, the subject has an A258T CFI mutation. In some embodiments, the subject has an A240G CFI mutation. In some embodiments, the subject has a T2031 CFI mutation. In some embodiments, the subject has an R187Q CFI mutation. In some embodiments, the subject has an R187Ter CFI mutation. In some embodiments, the subject has a G162D CFI mutation. In some embodiments, the subject has a V152M CFI mutation. In some embodiments, the subject has a G119R CFI mutation. In some embodiments, the subject is homozygous for at least one of the one or more CFI mutations. In some embodiments, the subject is heterozygous for at least one of the one or more CFI mutations. In some embodiments, the subject expresses a mutant CFI protein having reduced CFI activity as compared to a wild type CFI protein (e.g., a CFI protein having the amino acid sequence of SEQ ID NO: 1). In some embodiments, the CFI activity is the ability to cleave C3b to iC3b. In some embodiments, if a CFI protein having the CFI mutation were tested in a functional assay, the mutant CFI protein would display reduced CFI activity as compared to a wild type CFI protein (e.g., a CFI protein having the amino acid sequence of SEQ ID NO: 1). In some embodiments, the functional assay tests the ability of CFI to cleave C3b to iC3b. In some embodiments, the subject has atypical hemolytic uremic syndrome (aHUS). In some embodiments, the subject is suffering from a renal disease or complication. In some embodiments, the subject is a subject in whom it has been determined has one or more CFI mutations. In some embodiments, the subject is a subject in whom it has been determined has one or more CFI mutations selected from the group consisting of: G119R, L131R, V152M, G162D, R187Y, R187T, T2031, A240G, A258T, G287R, A300T, R317W, R339Q, V412M, and P553S. In some embodiments, the subject is a subject in whom it has been determined has one or more CFI mutations selected from the group consisting of: P553S, K441R, R339Q, R339Ter, R317Q, R317W, A300T, G287R, G261D, A258T, A240G, T2031, R187Q, R187Ter, G162D, V152M, and G119R. In some embodiments, the subject is a subject in whom it has been determined has a P553S CFI mutation. In some embodiments, the subject is a subject in whom it has been determined has a K441R CFI mutation. In some embodiments, the subject is a subject in whom it has been determined has an R339Q CFI mutation. In some embodiments, the subject is a subject in whom it has been determined has an R339Ter CFI mutation. In some embodiments, the subject is a subject in whom it has been determined has an R317Q CFI mutation. In some embodiments, the subject is a subject in whom it has been determined has an R317W CFI mutation. In some embodiments, the subject is a subject in whom it has been determined has an A300T CFI mutation. In some embodiments, the subject is a subject in whom it has been determined has a G287R CFI mutation. In some embodiments, the subject is a subject in whom it has been determined has a G261D CFI mutation. In some embodiments, the subject is a subject in whom it has been determined has an A258T CFI mutation. In some embodiments, the subject is a subject in whom it has been determined has an A240G CFI mutation. In some embodiments, the subject is a subject in whom it has been determined has a T2031 CFI mutation. In some embodiments, the subject is a subject in whom it has been determined has an R187Q CFI mutation. In some embodiments, the subject is a subject in whom it has been determined has an R187Ter CFI mutation. In some embodiments, the subject is a subject in whom it has been determined has a G162D CFI mutation. In some embodiments, the subject is a subject in whom it has been determined has a V152M CFI mutation. In some embodiments, the subject is a subject in whom it has been determined has a G119R CFI mutation. In some embodiments, the subject is a subject in whom it has been determined is homozygous for at least one of the one or more CFI mutations. In some embodiments, the subject is a subject in whom it has been determined is heterozygous for at least one of the one or more CFI mutations. In some embodiments, the CFI polypeptide or biologically active fragment and/or variant thereof is administered intravitreally.

### BRIEF DESCRIPTION OF THE DRAWINGS

FIGS. 1A-1D are Western blot gel images illustrating expression of various CFI mutant variants. FIG. 1A is a Western blot gel image illustrating expression of G119R CFI mutant variant. FIG. 1B is a Western blot gel image illustrating expression of A240G CFI mutant variant. FIG. 1C is a Western blot gel image illustrating expression of A300T CFI mutant variant. FIG. 1D is a Western blot gel image illustrating expression of P553S CFI mutant variant. In FIGS. 1A-1D, “Std” corresponds to the molecular weight standard, the arrow points to the mature form of CFI, and lane 3 contains conditioned medium from negative control cells that did not overexpress CFI constructs.

FIGS. 2A-2C are graphs from fluorescence cofactor assays. In each assay, increasing concentrations of wild type CFI or G119R CFI protein were mixed with a different cofactor (complement factor H (CFH) (FIG. 2A), MCP (membrane cofactor protein) (FIG. 2B), or complement receptor type 1 (CR1) (FIG. 2C)) and with 1-Anilino-8-Naphthalene Sulfonate (ANS)-labeled C3b, and relative fluorescent units (RFUs) were then measured over time and plotted against the concentration of CFI protein in μg/ml.

FIGS. 3A-3C are graphs from fluorescence cofactor assays. In each assay, increasing concentrations of wild type CFI or A240G CFI protein were mixed with a different cofactor (CFH (FIG. 3A), MCP (FIG. 3B), or CR1 (FIG. 3C)) and with ANS-labeled C3b, and relative fluorescent units (RFUs) were then measured over time and plotted against the concentration of CFI protein in μg/ml.

FIGS. 4A-4C are graphs from fluorescence cofactor assays. In each assay, increasing concentrations of wild type CFI or P553S CFI protein were mixed with a different cofactor (CFH (FIG. 4A), MCP (FIG. 4B), or CR1 (FIG. 4C)) and with ANS-labeled C3b, and relative fluorescent units (RFUs) were then measured over time and plotted against the concentration of CFI protein in μg/ml.

FIGS. 5A-5C are graphs from fluorescence cofactor assays. In each assay, increasing concentrations of wild type CFI or A300T CFI protein were mixed with a different cofactor (CFH (FIG. 5A), MCP (FIG. 5B), or CR1 (FIG. 5C)) and with ANS-labeled C3b, and relative fluorescent units (RFUs) were then measured over time and plotted against the concentration of CFI protein in μg/ml.

FIGS. 6A-6D are graphs from fluorescence cofactor assays. In each assay, increasing concentrations of CFH cofactor protein, VYE (CFH protein wherein amino acid residues at positions 62, 402, and 936 correspond to a valine, tyrosine, and glutamic acid, respectively) were mixed with wild type CFI or a CFI mutant (G119R (FIG. 6A), A240G (FIG. 6B), A300T (FIG. 6C), or P553S (FIG. 6D)) and with ANS-labeled C3b, and relative fluorescent units (RFUs) were then measured over time and plotted against the concentration of CFI protein in μg/ml.

### DETAILED DESCRIPTION OF THE DISCLOSURE

The disclosure provides compositions and methods for treating, preventing, or inhibiting a disease or disorder associated with one or more CFI mutations. In one aspect, the disclosure provides methods of treating, preventing, or inhibiting diseases of the eye associated with one or more CFI mutations by intraocularly (e.g., intravitreally) administering an effective amount of any of the recombinant CFI proteins or biologically active fragments and/or variants thereof.

A wide variety of diseases of the eye associated with one or more CFI mutations may be treated or prevented using the methods provided herein. Diseases of the eye that may be treated or prevented using the methods of the disclosure include but are not limited to, glaucoma, macular degeneration (e.g., age-related macular degeneration), diabetic retinopathies, inherited retinal degeneration such as retinitis pigmentosa, retinal detachment or injury and retinopathies (such as retinopathies that are inherited, induced by surgery, trauma, an underlying aetiology such as severe anemia, SLE, hypertension, blood dyscrasias, systemic infections, or underlying carotid disease, a toxic compound or agent, or photically).

### General Techniques

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N.Y. (2001); Ausubel et al., Current Protocols in Molecular Biology, John Wiley & Sons, N Y (2002); Harlow and Lane Using Antibodies: A Laboratory Manual, Cold Spring Harbor Laboratory Press, Cold Spring Harbor, N.Y. (1998); Coligan et al., Short Protocols in Protein Science, John Wiley & Sons, N Y (2003); Short Protocols in Molecular Biology (Wiley and Sons, 1999). The skilled worker would recognize many additional sources describing conventional techniques of molecular biology (including recombinant techniques), microbiology, cell biology, biochemistry and immunology.

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well-known and commonly used in the art. Standard techniques are used for chemical syntheses, and chemical analyses.

Throughout this specification and embodiments, the word “comprise,” or variations such as “comprises” or “comprising,” will be understood to imply the inclusion of a stated integer or group of integers but not the exclusion of any other integer or group of integers. It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the disclosure.

Exemplary methods and materials are described herein, although methods and materials similar or equivalent to those described herein can also be used in the practice or testing of the present disclosure. The materials, methods, and examples are illustrative only and not intended to be limiting.

### Definitions

The following terms, unless otherwise indicated, shall be understood to have the following meanings:

As used herein, “residue” refers to a position in a protein and its associated amino acid identity.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The terms “polypeptide,” “oligopeptide,” “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains. An amino acid mutation predicted to remove the entire C-terminal part of a protein starting at the site of the variant is described using “Ter.” For example, R187Ter indicates that arginine at position 187 is predicted to change to a stop codon (Ter).

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” or “percent (%) identical to” with respect to a reference polypeptide (or nucleotide) sequence is defined as the percentage of amino acid residues (or nucleic acids) in a candidate sequence that are identical with the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, a “host cell” includes an individual cell or cell culture that can be or has been a recipient for vector(s) for incorporation of polynucleotide inserts. In the alternative, the term “host cell” may refer to the target cell in which expression of the transgene is desired.

As used herein, “isolated molecule” (where the molecule is, for example, a polypeptide, a polynucleotide, or fragment thereof) is a molecule that by virtue of its origin or source of derivation (1) is not associated with one or more naturally associated components that accompany it in its native state, (2) is substantially free of one or more other molecules from the same species (3) is expressed by a cell from a different species, or (4) does not occur in nature.

As used herein, “purify,” and grammatical variations thereof, refers to the removal, whether completely or partially, of at least one impurity from a mixture containing the polypeptide and one or more impurities, which thereby improves the level of purity of the polypeptide in the composition (i.e., by decreasing the amount (ppm) of impurity(ies) in the composition).

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

The terms “patient,” “subject,” or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In some embodiments, the subject is a human that is at least 40, at least 45, at least 50, at least 55, at least 60, at least 65, at least 70, at least 75, at least 80, at least 85, at least 90, or at least 95 years of age.

In one embodiment, the subject has, or is at risk of developing a disease of the eye. A disease of the eye, includes, without limitation, AMD, retinitis pigmentosa, rod-cone dystrophy, Leber's congenital amaurosis, Usher's syndrome, Bardet-Biedl Syndrome, Best disease, retinoschisis, Stargardt disease (autosomal dominant or autosomal recessive), untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, glaucoma, or retinal vein occlusion. In another embodiment, the subject has, or is at risk of developing glaucoma, Leber's hereditary optic neuropathy, lysosomal storage disorder, or peroxisomal disorder. In another embodiment, the subject has shown clinical signs of a disease of the eye.

In some embodiments, the subject has, or is at risk of developing a renal disease or complication. In some embodiments, the renal disease or complication is associated with AMD or aHUS.

In some embodiments, the subject has, or is at risk of developing AMD or aHUS.

Clinical signs of a disease of the eye include, but are not limited to, decreased peripheral vision, decreased central (reading) vision, decreased night vision, loss of color perception, reduction in visual acuity, decreased photoreceptor function, and pigmentary changes. In one embodiment, the subject shows degeneration of the outer nuclear layer (ONL). In another embodiment, the subject has been diagnosed with a disease of the eye. In yet another embodiment, the subject has not yet shown clinical signs of a disease of the eye.

As used herein, the terms “prevent,” “preventing,” and “prevention” refer to the prevention of the recurrence or onset of, or a reduction in one or more symptoms of a disease or condition (e.g., a disease of the eye) in a subject as result of the administration of a therapy (e.g., a prophylactic or therapeutic agent). For example, in the context of the administration of a therapy to a subject for an infection, “prevent,” “preventing” and “prevention” refer to the inhibition or a reduction in the development or onset of a disease or condition (e.g., a disease of the eye), or the prevention of the recurrence, onset, or development of one or more symptoms of a disease or condition (e.g., a disease of the eye), in a subject resulting from the administration of a therapy (e.g., a prophylactic or therapeutic agent), or the administration of a combination of therapies (e.g., a combination of prophylactic or therapeutic agents).

“Treating” a condition or patient refers to taking steps to obtain beneficial or desired results, including clinical results. With respect to a disease or condition (e.g., a disease of the eye), treatment refers to the reduction or amelioration of the progression, severity, and/or duration of an infection (e.g., a disease of the eye or symptoms associated therewith), or the amelioration of one or more symptoms resulting from the administration of one or more therapies (including, but not limited to, the administration of one or more prophylactic or therapeutic agents).

“Administering” or “administration of” a substance, a compound or an agent to a subject can be carried out using one of a variety of methods known to those skilled in the art. For example, a compound or an agent can be administered intravitreally or subretinally. In particular embodiments, the compound or agent is administered intravitreally. In some embodiments, administration may be local. In other embodiments, administration may be systemic. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

As used herein, the term “ocular cells” refers to any cell in, or associated with the function of, the eye. The term may refer to any one or more of photoreceptor cells, including rod, cone and photosensitive ganglion cells, retinal pigment epithelium (RPE) cells, glial cells, Muller cells, bipolar cells, horizontal cells, amacrine cells. In one embodiment, the ocular cells are bipolar cells. In another embodiment, the ocular cells are horizontal cells. In another embodiment, the ocular cells are ganglion cells. In particular embodiments, the cells are RPE cells.

Each embodiment described herein may be used individually or in combination with any other embodiment described herein.

### Complement Factor I

The disclosure provides Complement Factor I (CFI) proteins or a biologically active fragment and/or variant thereof for use in any of the methods disclosed herein. The CFI proteins (or biologically active fragment or variant thereof) are described in greater detail below.

CFI is a serine protease which degrades activated complement factors (e.g. C4b and C3b) in the presence of cofactors (e.g. C4b-binding protein, factor H, complement receptor 1, and membrane cofactor protein). CFI is composed of a light chain and heavy chain covalently linked by a disulfide bond. The light chain comprises the serine protease domain, whereas the heavy chain comprises the factor I (FI) and membrane attack complex domain (FIMAC); the CD5-like (CD5) domain, also known as a scavenger receptor cysteine-rich (SRCR) domain; and two low-density lipoprotein receptor Class A domains (LDLRA1 and LDLRA2).

In some embodiments, the CFI polypeptide or biologically active fragment and/or variant thereof comprises a heavy chain and a light chain. In some embodiments, the CFI polypeptide or biologically active fragment and/or variant thereof comprises a disulfide bond between the heavy chain and light chain. In some embodiments, the CFI polypeptide or biologically active fragment and/or variant thereof comprises a FIMAC domain. In some embodiments, the CFI polypeptide or biologically active fragment and/or variant thereof comprises a Scavenger Receptor Cysteine Rich (SRCR) domain. In some embodiments, the CFI polypeptide or biologically active fragment and/or variant thereof comprises at least one LDL receptor Class A domain. In some embodiments, the CFI polypeptide or biologically active fragment and/or variant thereof comprises two LDL receptor Class A domains. In some embodiments, the CFI polypeptide or biologically active fragment and/or variant thereof comprises a serine protease domain. In some embodiments, the CFI polypeptide or biologically active fragment and/or variant thereof comprises a FIMAC domain, a Scavenger Receptor Cysteine Rich (SRCR) domain, and two LDL receptor Class A domains. In some embodiments, the CFI polypeptide or biologically active fragment and/or variant thereof is capable of cleaving C3b and C4b proteins.

CFI is only able to degrade C4b and C3b in the presence of cofactors such as CFH. When appropriate levels of CFI and its cofactors are present, C3b is broken down to iC3b resulting in three downstream effects: (i) decreased assembly of the C3 convertases; (ii) prevention of runaway C3 consumption; and (iii) generation of the C3b cleavage fragments that go on to bind specific complement receptors and are involved in opsonization and triggering of the adaptive immune response. In some embodiments, the CFI protein is capable of inducing one or more of the listed downstream effects when administered to a subject. In such embodiments, it is expected that the necessary cofactors for CFI activity are either already present in the subject or separately administered to the subject.

In some embodiments, the CFI polypeptide or biologically active fragment and/or variant thereof is capable of inhibiting the assembly of the C3 convertase enzyme. In some embodiments, the CFI polypeptide or biologically active fragment and/or variant thereof is capable of inhibiting the assembly of the C5 convertase enzyme. In some embodiments, the CFI polypeptide or biologically active fragment and/or variant thereof is capable of inhibiting the assembly of C3 and C5 convertase enzymes. In some embodiments, the CFI polypeptide or biologically active fragment and/or variant thereof is capable of cleaving C3b to form iC3b.

In some embodiments, the CFI polypeptide comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 1, or a biologically active fragment thereof. In some embodiments, the CFI polypeptide comprises an amino acid sequence that is at least 90% identical to the amino acid sequence of SEQ ID NO: 1, or a biologically active fragment thereof. In some embodiments, the CFI polypeptide comprises an amino acid sequence that is at least 95% identical to the amino acid sequence of SEQ ID NO: 1, or a biologically active fragment thereof. In some embodiments, the CFI polypeptide comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 2, or a biologically active fragment thereof. In some embodiments, the CFI polypeptide comprises an amino acid sequence that is at least 90% identical to the amino acid sequence of SEQ ID NO: 2, or a biologically active fragment thereof. In some embodiments, the CFI polypeptide comprises an amino acid sequence that is at least 95% identical to the amino acid sequence of SEQ ID NO: 2, or a biologically active fragment thereof.

In some embodiments, the CFI polypeptide is encoded by a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of any one of SEQ ID NOs: 3-6 or a biologically thereof.

In some embodiments, the CFI polypeptide or biologically active fragment and/or variant thereof of the present disclosure is capable of inducing any one or more of the effects listed above with regard to a wild type CFI protein. For example, in some embodiments, the CFI polypeptide or biologically active fragment and/or variant thereof of the present disclosure is capable of inhibiting the assembly of the C3 convertase enzyme. In some embodiments, the CFI polypeptide or biologically active fragment and/or variant thereof of the present disclosure is capable of inhibiting the assembly of the C5 convertase enzyme. In some embodiments, the CFI polypeptide or biologically active fragment and/or variant thereof of the present disclosure is capable of inhibiting the assembly of C3 and C5 convertase enzymes. In some embodiments, the CFI polypeptide or biologically active fragment and/or variant thereof of the present disclosure is capable of cleaving C3b to form iC3b. In some embodiments, the CFI polypeptide or biologically active fragment and/or variant thereof of the present disclosure is at least 200, at least 250, at least 300, at least 350, at least 400, at least 450, at least 500, at least 550, or at least 583 amino acids in length. In some embodiments, CFI polypeptide or biologically active fragment and/or variant thereof of the present disclosure is between 100-583, between 200-583, between 300-583, between 400-583, between 500-583, between 100-200, between 100-300, between 100-400, between 100-500, between 200-300, between 200-400, between 200-500, between 300-400, between 300-500, or between 400-500 amino acids in length. In some embodiments, the CFI polypeptide or biologically active fragment and/or variant thereof of the present disclosure comprises at least 100, at least 150, at least 200, at least 250, at least 300, at least 350, at least 400, at least 450, at least 500, at least 550, or at least 582 consecutive amino acids from a sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 1. In some embodiments, the CFI polypeptide or biologically active fragment and/or variant thereof of the present disclosure comprises at least 100, at least 150, at least 200, at least 250, at least 300, at least 350, at least 400, at least 450, at least 500, or at least 550 consecutive amino acids of SEQ ID NO: 1. In some embodiments, the CFI polypeptide or biologically active fragment and/or variant thereof of the present disclosure comprises a FIMAC domain. In some embodiments, the CFI polypeptide or biologically active fragment and/or variant thereof of the present disclosure comprises a Scavenger Receptor Cysteine Rich (SRCR) domain. In some embodiments, the CFI polypeptide or biologically active fragment and/or variant thereof of the present disclosure comprises at least one LDL receptor Class A domain. In some embodiments, the CFI polypeptide or biologically active fragment and/or variant thereof of the present disclosure comprises two LDL receptor Class A domains. In some embodiments, the CFI polypeptide or biologically active fragment and/or variant thereof of the present disclosure comprises a serine protease domain. In some embodiments, the CFI polypeptide or biologically active fragment and/or variant thereof of the present disclosure comprises a FIMAC domain, a Scavenger Receptor Cysteine Rich (SRCR) domain, and two LDL receptor Class A domains. In some embodiments, the CFI polypeptide or biologically active fragment and/or variant thereof of the present disclosure is capable of cleaving C3b and C4b proteins.

In some embodiments, the CFI polypeptide or biologically active fragment and/or variant thereof is a precursor CFI polypeptide or biologically active fragment and/or variant thereof. In some embodiments, the precursor CFI polypeptide or biologically active fragment and/or variant thereof is processed to a mature CFI polypeptide or biologically active fragment and/or variant thereof after administration to a subject. In some embodiments, the CFI polypeptide or biologically active fragment and/or variant thereof is a mature CFI polypeptide or biologically active fragment and/or variant thereof. In some embodiments, the CFI polypeptide or biologically active fragment and/or variant thereof has been exposed to a protease (e.g., a furin protease) to obtain the mature CFI polypeptide or biologically active fragment and/or variant thereof.

In certain embodiments, any of the CFI polypeptides or biologically active fragments and/or variants disclosed herein may further comprise post-translational modifications in addition to any that are naturally present in the native polypeptides. Such modifications include, but are not limited to, acetylation, carboxylation, glycosylation, phosphorylation, lipidation, pegylation (polyethylene glycol) and acylation. As a result, the modified polypeptides may contain non-amino acid elements, such as polyethylene glycols, lipids, mono- or poly-saccharides, and phosphates. Effects of such non-amino acid elements on the functionality of a polypeptide may be tested as described herein for other polypeptide variants. When a polypeptide is produced in cells by cleaving a nascent form of the polypeptide, post-translational processing may also be important for correct folding and/or function of the protein. Different cells have specific cellular machinery and characteristic mechanisms for such post-translational activities and may be chosen to ensure the correct modification and processing of the polypeptides.

### Pharmaceutical Compositions and Routes of Administration

Also provided herein are pharmaceutical compositions comprising any of the CFI polypeptides or biologically active fragments and/or variants thereof disclosed herein, and a pharmaceutically acceptable carrier. The pharmaceutical compositions may be suitable for any mode of administration described herein; for example, by intravitreal administration.

In some embodiments, the composition comprises a CFI polypeptide or biologically active fragment and/or variant thereof. In particular embodiments, the composition does not comprise a polypeptide (or a nucleic acid encoding a polypeptide) that processes (e.g., cleaves) a CFI polypeptide. In some embodiments, the processing polypeptide is a protease. In some embodiments, the protease is furin.

In some embodiments, the pharmaceutical compositions comprising a CFI polypeptide or biologically active fragment and/or variant thereof described herein and a pharmaceutically acceptable carrier is suitable for administration to a human subject. Such carriers are well known in the art (see, e.g., Remington's Pharmaceutical Sciences, 15th Edition, pp. 1035-1038 and 1570-1580). In some embodiments, the pharmaceutical compositions comprising a CFI polypeptide or biologically active fragment and/or variant thereof described herein and a pharmaceutically acceptable carrier is suitable for ocular injection. In some embodiments, the pharmaceutical composition is suitable for intravitreal injection. In some embodiments, the pharmaceutical composition is suitable for subretinal delivery. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms. The compositions are generally formulated as sterile and substantially isotonic solution.

In one embodiment, the CFI polypeptide or biologically active fragment and/or variant thereof is formulated into a pharmaceutical composition intended for subretinal or intravitreal injection. Such formulation involves the use of a pharmaceutically and/or physiologically acceptable vehicle or carrier, particularly one suitable for administration to the eye, e.g., by subretinal or intravitreal injection, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the CFI polypeptide or biologically active fragment and/or variant thereof is to be stored long-term, it may be frozen in the presence of glycerol or Tween20. In another embodiment, the pharmaceutically acceptable carrier comprises a surfactant, such as perfluorooctane (Perfluoron liquid).

In certain embodiments of the methods described herein, the pharmaceutical composition described above is administered to the subject by subretinal injection. In other embodiments, the pharmaceutical composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parenteral routes of administration. Routes of administration may be combined, if desired. In certain embodiments, the pharmaceutical compositions of the disclosure are administered after administration of an initial loading dose of the CFI polypeptide or biologically active fragment and/or variant thereof.

In some embodiments, any of the CFI polypeptides or biologically active fragments and/or variants thereof or pharmaceutical compositions disclosed herein are administered to a patient such that they target cells of any one or more layers or regions of the retina or macula. For example, the compositions disclosed herein target cells of any one or more layers of the retina, including the inner limiting membrane, the nerve fiber layer, the ganglion cell layer (GCL), the inner plexiform layer, the inner nuclear layer, the outer plexiform layer, the outer nuclear layer, the external limiting membrane, the layer of rods and cones, or the retinal pigment epithelium (RPE). In some embodiments, the compositions disclosed herein target glial cells of the GCL, Muller cells, and/or retinal pigment epithelial cells. In some embodiments, the compositions disclosed herein targets cells of any one or more regions of the macula including, for example, the umbo, the foveolar, the foveal avascular zone, the fovea, the parafovea, or the perifovea. In some embodiments, the route of administration does not specifically target neurons. In some embodiments, the route of administration is chosen such that it reduces the risk of retinal detachment in the patient (e.g., intravitreal rather than subretinal administration). In some embodiments, intravitreal administration is chosen if any of the CFI polypeptides or biologically active fragments and/or variants thereof disclosed herein is to be administered to an elderly adult (e.g., at least 60 years of age). In particular embodiments, any of the CFI polypeptides or biologically active fragments and/or variants thereof or pharmaceutical compositions disclosed herein are administered to a subject intravitreally. Procedures for intravitreal injection are known in the art (see, e.g., Peyman, G. A., et al. (2009) Retina 29(7):875-912 and Fagan, X. J. and Al-Qureshi, S. (2013) Clin. Experiment. Ophthalmol. 41(5):500-7). Briefly, a subject for intravitreal injection may be prepared for the procedure by pupillary dilation, sterilization of the eye, and administration of anesthetic. Any suitable mydriatic agent known in the art may be used for pupillary dilation. Adequate pupillary dilation may be confirmed before treatment. Sterilization may be achieved by applying a sterilizing eye treatment, e.g., an iodide-containing solution such as Povidone-Iodine (BETADINE®). A similar solution may also be used to clean the eyelid, eyelashes, and any other nearby tissues (e.g., skin). Any suitable anesthetic may be used, such as lidocaine or proparacaine, at any suitable concentration. Anesthetic may be administered by any method known in the art, including without limitation topical drops, gels or jellies, and subconjunctival application of anesthetic. Prior to injection, a sterilized eyelid speculum may be used to clear the eyelashes from the area. The site of the injection may be marked with a syringe. The site of the injection may be chosen based on the lens of the patient. For example, the injection site may be 3-3.5 mm from the limbus in pseudophakic or aphakic patients, and 3.5 mm-4 mm from the limbus in phakic patients. The patient may look in a direction opposite the injection site. During injection, the needle may be inserted perpendicular to the sclera and pointed to the center of the eye. The needle may be inserted such that the tip ends in the vitreous, rather than the subretinal space. Any suitable volume known in the art for injection may be used. After injection, the eye may be treated with a sterilizing agent such as an antibiotic. The eye may also be rinsed to remove excess sterilizing agent.

Furthermore, in certain embodiments it is desirable to perform non-invasive retinal imaging and functional studies to identify areas of specific ocular cells to be targeted for therapy. In these embodiments, clinical diagnostic tests are employed to determine the precise location(s) for one or more subretinal injection(s). These tests may include ophthalmoscopy, electroretinography (ERG) (particularly the b-wave measurement), perimetry, topographical mapping of the layers of the retina and measurement of the thickness of its layers by means of confocal scanning laser ophthalmoscopy (cSLO) and optical coherence tomography (OCT), topographical mapping of cone density via adaptive optics (AO), functional eye exam, etc.

These, and other desirable tests, are described in International Patent Application No. PCT/US2013/022628. In view of the imaging and functional studies, in some embodiments, one or more injections are performed in the same eye in order to target different areas of retained bipolar cells. The volume and concentration of the CFI polypeptide or biologically active fragment and/or variant thereof for each injection is determined individually, as further described below, and may be the same or different from other injections performed in the same, or contralateral, eye. In another embodiment, a single, larger volume injection is made in order to treat the entire eye. In one embodiment, the volume and concentration of the CFI polypeptide or biologically active fragment and/or variant thereof composition is selected so that only a specific region of ocular cells is impacted. In another embodiment, the volume and/or concentration of the CFI polypeptide or biologically active fragment and/or variant thereof composition is a greater amount, in order reach larger portions of the eye, including non-damaged ocular cells.

The composition may be delivered in a volume of from about 0.1 μL to about 1 mL, including all numbers within the range, depending on the size of the area to be treated, the route of administration, and the desired effect of the method. In some embodiments, the volume is between 25 μL-100 μL. In some embodiments, the volume is between 40 μL-60 μL. In one embodiment, the volume is about 50 μL. In another embodiment, the volume is about 70 μL. In a preferred embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL. It is desirable that the lowest effective concentration of CFI polypeptide or biologically active fragment and/or variant thereof be utilized in order to reduce the risk of undesirable effects, such as toxicity, retinal dysplasia and detachment. Still other dosages and administration volumes in these ranges may be selected by the attending physician, taking into account the physical state of the subject, preferably human, being treated, the age of the subject, the particular ocular disorder and the degree to which the disorder, if progressive, has developed. For extra-ocular delivery, the dosage will be increased according to the scale-up from the retina.

### Methods of Treatment/Prophylaxis

In some embodiments, the disclosure provides a method for treating a subject having a disease or disorder, wherein the subject has one or more CFI mutations. A subject “has” a CFI mutation if DNA from a sample (e.g., a blood sample or a sample from the patient's eye) from the subject is determined to carry one or more CFI mutations. In some embodiments, any of the methods disclosed herein are for treating a subject in whom it has been determined to have one or more CFI mutations. In some embodiments, the presence or absence of any of the CFI mutations disclosed herein is determined by genetic testing.

Described herein are various methods of preventing, treating, arresting progression of or ameliorating the ocular disorders and retinal changes associated therewith. Generally, the methods include administering to a mammalian subject in need thereof, an effective amount of a composition comprising a CFI polypeptide or biologically active fragment and/or variant thereof as described above, and a pharmaceutically acceptable carrier. Any of the CFI polypeptides or biologically active fragments and/or variants thereof described herein are useful in the methods described below.

In some embodiments, CFI polypeptide or biologically active fragment and/or variant thereof are administered locally to the cells in the retina for treating diseases such as leber congenital amaurosis (LCA), retinitis pigmentosa, and age-related macular degeneration. The cells that will be the treatment target in these diseases are either the photoreceptor cells in the retina or the cells of the RPE underlying the neurosensory retina. Delivering CFI polypeptide or biologically active fragment and/or variant thereof to these cells may be by injection into the subretinal space between the retina and the RPE. In some embodiments, the disclosure provides methods to deliver CFI polypeptide or biologically active fragment and/or variant thereof to cells of the retina.

In a certain aspect, the disclosure provides a method of treating a subject having age-related macular degeneration (AMD), comprising the step of administering to the subject any of the CFI polypeptides or biologically active fragments and/or variants thereof of the disclosure. In certain embodiments, the pharmaceutical compositions of the disclosure comprise a pharmaceutically acceptable carrier. In certain embodiments, the pharmaceutical compositions of the disclosure comprise PBS. In certain embodiments, the pharmaceutical compositions of the disclosure comprise pluronic. In certain embodiments, the pharmaceutical compositions of the disclosure comprise PBS, NaCl and pluronic. In certain embodiments, the CFI polypeptides or biologically active fragments and/or variants thereof are administered by intravitreal injection in a solution of PBS with additional NaCl and pluronic.

In some embodiments, any of the CFI polypeptides or biologically active fragments and/or variants thereof disclosed herein is administered to cell(s) or tissue(s) in a test subject. In some embodiments, the cell(s) or tissue(s) in the test subject express less CFI, or less functional CFI, than expressed in the same cell type or tissue type in a reference control subject or population of reference control subjects. In some embodiments, the reference control subject is of the same age and/or sex as the test subject. In some embodiments, the reference control subject is a healthy subject, e.g., the subject does not have a disease or disorder of the eye. In some embodiments, the reference control subject does not have a disease or disorder of the eye associated with activation of the complement cascade. In some embodiments, the reference control subject does not have macular degeneration. In some embodiments, the eye or a specific cell type of the eye (e.g., cells in the foveal region) in the test subject expresses at least 95%, at least 90%, at least 80%, at least 70%, at least 60%, at least 50%, at least 40%, at least 30%, at least 20%, at least 10%, at least 5%, or at least 1% less CFI or functional CFI as compared to the levels in the reference control subject or population of reference control subjects. In some embodiments, the eye or a specific cell type of the eye (e.g., cells in the foveal region) in the test subject express CFI protein having any one or more of the CFI mutations disclosed herein. In some embodiments, the eye or a specific cell type of the eye (e.g., cells in the foveal region) in the reference control subject do not express a CFI protein having any of the CFI mutations disclosed herein. In some embodiments, administration of any of the CFI polypeptides or biologically active fragments and/or variants thereof disclosed herein to the cell(s) or tissue(s) of the test subject results in an increase in levels of functional CFI protein. In some embodiments, administration of any of the CFI polypeptides or biologically active fragments and/or variants thereof disclosed herein to the cell(s) or tissue(s) of the test subject results in an increase in levels of functional CFI protein such that the increased levels are within 90%, within 80%, within 70%, within 60%, within 50%, within 40%, within 30%, within 20%, within 10%, within 5%, or within 1% of, or are the same as, the levels functional CFI protein expressed by the same cell type or tissue type in the reference control subject or population of reference control subjects. In some embodiments, administration of any of the CFI polypeptides or biologically active fragments and/or variants thereof disclosed herein to the cell(s) or tissue(s) of the test subject results in an increase in levels of functional CFI protein, but the increased levels of functional CFI protein do not exceed the levels of functional CFI protein expressed by the same cell type or tissue type in the reference control subject or population of reference control subjects. In some embodiments, administration of any of the CFI polypeptides or biologically active fragments and/or variants thereof disclosed herein to the cell(s) or tissue(s) of the test subject results in an increase in levels functional CFI protein, but the increased levels of functional CFI protein exceed the levels functional CFI protein by no more than 1%, no more than 5%, no more than 10%, no more than 20%, no more than 25%, no more than 30%, no more than 40%, no more than 50%, no more than 60%, no more than 70%, no more than 80%, no more than 90%, or 100% of the levels expressed by the same cell type or tissue type in the reference control subject or population of reference control subjects.

In some embodiments, any of the treatment and/or prophylactic methods disclosed herein are applied to a subject. In some embodiments, the subject is a mammal. In some embodiments, the subject is a human. In some embodiments, the human is an adult. In some embodiments, the human is an elderly adult. In some embodiments, the human is at least 40, at least 45, at least 50, at least 55, at least 60, at least 65, at least 70, at least 75, at least 80, at least 85, at least 90, or at least 95 years of age. In particular embodiments, the human is at least 60 or at least 65 years of age.

In some embodiments, any of the treatment and/or prophylactic methods disclosed herein is for use in treatment of a patient having one or more mutations that causes macular degeneration (AMD) or that increases the likelihood that a patient develops AMD. In some embodiments, any of the treatment and/or prophylactic methods disclosed herein is for use in treatment of a patient having one or more mutations that causes atypical hemolytic uremic syndrome (aHUS) or that increases the likelihood that a patient develops aHUS. In some embodiments, the one or more mutations are in the patient's CFI gene. In some embodiments, the subject has a loss-of-function mutation in the subject's CFI gene.

In some embodiments, any of the treatment and/or prophylactic methods disclosed herein is for use in treatment of a patient having one or more mutations in the patient's CFI gene. In some embodiments, the treatment and/or prophylactic method is for use in treating a patient in whom it has been determined has one or more of any of the CFI mutations disclosed herein. In some embodiments, the patient has a mutation in one or more of the FIMAC, CD5, L1, L1-Ca binding, L1-disulfide bond, L2, L2-Ca binding, serine protease, or serine protease active site domains. In some embodiments, the patient has one or more mutations in the disulphide bond sites in the CFI protein. In some embodiments, the mutation is one or more of the mutations selected from the group consisting of: E548Q, V412M, A431T, A431S, K441R, P553S, A240G, A258T, G119R, G261D, R2021, T300A, T2031, V152M, R317W, G287R, E554V, I340T, G162D, P50A, Y206N, D310E, H418L, p.(Tyr411Stop), p.(Arg187Stop), R474Q, Y459S, R187Q, R339Q, G263V, p.(Arg339Stop), D477H, p.(Ile357Met), P64L, E109A, G125R, N1771, F198L, S221Y, D224N, C229R, V230M, G248E, G280D, A356P, V20I, Y369S, W374C, R389H, W399R, C467R, G487C, I492L, G500R, R502C, W541*, V543A, Q580*, V355M, I578T, R474*, R406H, D44N, p.(Arg406Cys), D403N, 1416L, G328R, G512S, p.(Gly542Ser), p.(Cys106Arg), V127A, p.(Ile55Phe), H40R, C54R, C54*, V184M, G362A, Q462H, N536K, R317Q, p.(His 183Arg), p.(Ile306Val), p.(Gly342Glu), p.(Asp429Glu), R448H, D519N, S493R, R448C, K338Q, G104R, C259R, G372S, A360V, E290A, V213F, F13V, Y514Ter, V396A, E303Q, H401Q, 1306T, E479G, c.772+1G>T, F498L, Y411H, S24T, C255Y, R168S, Q228R, V469I, Q250K, Y241C, G232V, G248R, G110R, E109K, N422D, C550R, G242AfsTer9, R345G, N428MfsTer5, C550WfsTer17, V341E, N428S, H334P, W51R, A452S, T72S, T72S, V558I, E445G, C444Y, L351I, G261S, M1381, A563S, G263AfsTer37, K142E, c.658+2T>C, G205D, T197A, G188V, A378V, L376P, C365Y, M147V, Q161Ter, G439R, G269S, R201S, P576S, Y65H, c.907+1G>Aâ€, Y22C, 1407T, M204V, A384T, G516V, R336G, F139V, L4H, K117E, V489I, P402L, G547R, A346T, S326P, I126T, D283G, S298F, loss of Metl, Ter584QextTer24, C521Y, R168G, S457P, A423E, L34V, A452T, K442E, N245K, D173N, K267E, S146R, E302K, G295V, V299L, K111N, S113N, F17V, Q391E, H14L, T394I, c.659-2A>G, A511V, E303K, D398G, Ter584KextTer24, V583A, A163T, H118Q, A309S, T23I, G473R, V530I, E26Ter, K497N, S496C, S496T, L491R, V412E, F417S, S570G, D465G, E124K, D567V, G557D, E548G, W546G, V543I, N464K, P463A, N564S, K561E, E445D, C444G, D443H, E434KfsTer2, 1430T, I244S, I244V, c328+1G>A, R345Q, S175F, N331KfsTer46, C327R, K1301, Q260E, P96S, 1140T, T1371, D135G, K69E, G57D, G371V, G367A, N279S, Y276C, G269C, E190D, T300A, G261D, N151S, R406H, V152M, G362A, E554V, S570T, I340T, K441R, T2031, Y206N, G328R, T107A, P553S, G287R, N70T, P50A, R406C, R187Q, G119R, 0.1429+1G>C, D477H, N1771, V129A, I55V, W399R, G500R, I492L, R339Ter, I357M, R474Q, D44N, D403N, R474Ter, R317W, G512S, R339Q, A356P, R187Ter, 1416L, R317L, R389H, 1306V, D224Y, R317Q, A258T, Q580Tet, H418L, I578T, G542S, P64L, C106R, Y369S, Q462H, A240G, H183R, R502G, H40R and G162D. In particular embodiments, the mutation is any one of the mutations selected from the group consisting of: G119R, L131R, V152M, G162D, R187Y, R187T, T2031, A240G, A258T, G287R, A300T, R317W, R339Q, V412M, and P553S. In some embodiments, any of the CFI mutant amino acid positions described herein correspond to the wild type amino acid CFI sequence of SEQ ID NO: 1.

In some embodiments, the subject has been determined to have one or more CFI mutations, wherein one or more CFI mutations are selected from the group consisting of: G119R, L131R, V152M, G162D, R187Y, R187T, T2031, A240G, A258T, G287R, A300T, R317W, R339Q, V412M, and P553S. In some embodiments, the patient has any one of the following mutations: G119R, P553S, K441R, R339Q, R339Ter, R317Q, R317W, A300T, G287R, G261D, A258T, A240G, T2031, R187Q, R187Ter, G162D, or V152M. In some embodiment, the patient has a P553S mutation. In some embodiments, the patient has a K441R mutation. In some embodiments, the patient has an R339Q mutation. In some embodiments, the patient has an R339Ter mutation. In some embodiments, the patient has an R317Q mutation. In some embodiments, the patient has an R317W mutation. In some embodiments, the patient has an A300T mutation. In some embodiments, the patient has a G287R mutation. In some embodiments, the patient has a G261D mutation. In some embodiments, the patient has an A258T mutation. In some embodiments, the patient has an A240G mutation. In some embodiments, the patient has a T2031 mutation. In some embodiments, the patient has an R187Q mutation. In some embodiments, the patient has an R187Ter mutation. In some embodiments, the patient has a G162D mutation. In some embodiments, the patient has a V152M mutation. In some embodiments, the patient has a G119R mutation. In some embodiments, any of the CFI mutant amino acid positions described herein correspond to the wild type amino acid CFI sequence of SEQ ID NO: 1. In some embodiments, the patient has one or more of the following mutations: G119R, P553S, K441R, R339Q, R339Ter, R317Q, R317W, A300T, G287R, G261D, A258T, A240G, T2031, R187Q, R187Ter, G162D, or V152M.

Documents referencing some of the CFI mutations disclosed herein include: Saksens et al., 2016, JAMA Ophthalmol, 134(3):287-293; Nilsson et al., 2010, Eur. J. Immunol., 40:172-185; Nilsson et al., 2007, Molecular Immunol., 44:1835-1844; Kavanagh et al., 2015, Human Molecular Genetics, 24(13):3861-3870; Kavanagh et al., 2008, Molecular Immunology, 45:95-105; Geerlings et al., 2018, Clinical Genetics, 94:330-338; Geerlings et al., 2017, JAMA Ophthalmol, 135(1): 39-46; Fritsche et al., 2016, Nat. Genet., 48(2):134-143; Cayci et al., 2012, Pediatr Nephrol., 27:2327-2331; Caprioli et al., 2006, Blood, 108(4):1267-1279; Bienaime et al., 2010, Kidney International, 77:334-349; Alexander et al., 2014, Molecular Vision, 20:1253-57; Seddon et al., 2013, Nat. Genet., 45(11):1366-1370; and Van de Ven et al., 2013, Nat. Genet., 45(7):813-819.

In some embodiments, any of the CFI mutant amino acid positions described herein correspond to the wild type amino acid CFI sequence of SEQ ID NO: 1. In some embodiments, if the subject has a CFI mutation that results in reduced CFI activity as compared to a wild type CFI protein, then the subject is administered a greater amount of any of the CFI proteins or biologically active fragments and/or variants disclosed herein. In some embodiments, one or more CFI mutations disclosed herein reduce CFI activity as compared to a wild type CFI protein. In some embodiments, the mutant CFI protein displays reduced CFI activity as compared to a wild type CFI protein in a functional assay.

In some embodiments, the patient is homozygous for any of the mutations disclosed herein. In some embodiments, the patient is heterozygous for any of the mutations disclosed herein. In particular embodiments, the patient expresses a mutant CFI protein, wherein the mutant CFI protein has reduced CFI activity as compared to a wild type CFI protein (e.g., a CFI protein having the amino acid sequence of SEQ ID NO: 1). In some embodiments, the CFI activity is the ability to cleave C3b to iC3b. In some embodiments, if the mutant CFI protein were tested in a functional assay, the mutant CFI protein would display reduced CFI activity as compared to a wild type CFI protein (e.g., a CFI protein having the amino acid sequence of SEQ ID NO: 1). In some embodiments, the functional assay tests the ability of CFI to cleave C3b to iC3b (see, e.g., Example 1 for a representative assay testing the ability of CFI to cleave C3b to iC3b). Examples of CFI mutants associated with reduced CFI activity (e.g., reduce ability to cleave C3b to iC3b) include G119R, A240G, or P553S CFI mutants. See, e.g., Example 1.

In some embodiments, any of the methods disclosed herein are for treating a subject carrying one or more of any of the mutations disclosed herein.

In some embodiments, any of the CFI polypeptides or biologically active fragments and/or variants thereof disclosed herein are for use in treating a renal disease or complication. In some embodiments, the renal disease or complication is associated with AMD in the patient. In some embodiments, the renal disease or complication is associated with aHUS in the patient.

The retinal diseases described above are associated with various retinal changes. These may include a loss of photoreceptor structure or function; thinning or thickening of the outer nuclear layer (ONL); thinning or thickening of the outer plexiform layer (OPL); disorganization followed by loss of rod and cone outer segments; shortening of the rod and cone inner segments; retraction of bipolar cell dendrites; thinning or thickening of the inner retinal layers including inner nuclear layer, inner plexiform layer, ganglion cell layer and nerve fiber layer; opsin mislocalization; overexpression of neurofilaments; thinning of specific portions of the retina (such as the fovea or macula); loss of ERG function; loss of visual acuity and contrast sensitivity; loss of optokinetic reflexes; loss of the pupillary light reflex; and loss of visually guided behavior. In one embodiment, a method of preventing, arresting progression of or ameliorating any of the retinal changes associated with these retinal diseases is provided. As a result, the subject's vision is improved, or vision loss is arrested and/or ameliorated.

In a particular embodiment, a method of preventing, arresting progression of or ameliorating vision loss associated with an ocular disorder in the subject is provided. Vision loss associated with an ocular disorder refers to any decrease in peripheral vision, central (reading) vision, night vision, day vision, loss of color perception, loss of contrast sensitivity, or reduction in visual acuity.

In any of the methods described herein, the targeted cell may be an ocular cell. In one embodiment, the targeted cell is a glial cell. In one embodiment, the targeted cell is an RPE cell. In another embodiment, the targeted cell is a photoreceptor. In another embodiment, the photoreceptor is a cone cell. In another embodiment, the targeted cell is a Muller cell. In another embodiment, the targeted cell is a bipolar cell. In yet another embodiment, the targeted cell is a horizontal cell. In another embodiment, the targeted cell is an amacrine cell. In still another embodiment, the targeted cell is a ganglion cell. In still another embodiment, the gene may be expressed and delivered to an intracellular organelle, such as a mitochondrion or a lysosome.

As used herein “photoreceptor function loss” means a decrease in photoreceptor function as compared to a normal, non-diseased eye or the same eye at an earlier time point. In some embodiments, any of the methods disclosed herein may be used to increase photoreceptor function in a subject in need thereof. As used herein, “increase photoreceptor function” means to improve the function of the photoreceptors or increase the number or percentage of functional photoreceptors as compared to a diseased eye (having the same ocular disease), the same eye at an earlier time point, a non-treated portion of the same eye, or the contralateral eye of the same patient. Photoreceptor function may be assessed using the functional studies described above and in the examples below, e.g., ERG or perimetry, which are conventional in the art.

For each of the described methods, the treatment may be used to prevent the occurrence of retinal damage or to rescue eyes having mild or advanced disease. As used herein, the term “rescue” means to prevent progression of the disease to total blindness, prevent spread of damage to uninjured ocular cells, improve damage in injured ocular cells, or to provide enhanced vision. In one embodiment, the composition is administered before the disease becomes symptomatic or prior to photoreceptor loss. By symptomatic is meant onset of any of the various retinal changes described above or vision loss. In another embodiment, the composition is administered after disease becomes symptomatic. In yet another embodiment, the composition is administered after initiation of photoreceptor loss. In another embodiment, the composition is administered after outer nuclear layer (ONL) degeneration begins. In some embodiments, it is desirable that the composition is administered while bipolar cell circuitry to ganglion cells and optic nerve remains intact.

In another embodiment, the composition is administered after initiation of photoreceptor loss. In yet another embodiment, the composition is administered when less than 90% of the photoreceptors are functioning or remaining, as compared to a non-diseased eye. In another embodiment, the composition is administered when less than 80% of the photoreceptors are functioning or remaining. In another embodiment, the composition is administered when less than 70% of the photoreceptors are functioning or remaining. In another embodiment, the composition is administered when less than 60% of the photoreceptors are functioning or remaining. In another embodiment, the composition is administered when less than 50% of the photoreceptors are functioning or remaining. In another embodiment, the composition is administered when less than 40% of the photoreceptors are functioning or remaining. In another embodiment, the composition is administered when less than 30% of the photoreceptors are functioning or remaining. In another embodiment, the composition is administered when less than 20% of the photoreceptors are functioning or remaining. In another embodiment, the composition is administered when less than 10% of the photoreceptors are functioning or remaining. In one embodiment, the composition is administered only to one or more regions of the eye. In another embodiment, the composition is administered to the entire eye.

In another embodiment, the method includes performing functional and imaging studies to determine the efficacy of the treatment. These studies include ERG and in vivo retinal imaging, as described in the examples below. In addition visual field studies, perimetry and microperimetry, pupillometry, mobility testing, visual acuity, contrast sensitivity, color vision testing may be performed.

In yet another embodiment, any of the above described methods is performed in combination with another, or secondary, therapy. The therapy may be any now known, or as yet unknown, therapy which helps prevent, arrest or ameliorate any of the described retinal changes and/or vision loss. In one embodiment, the secondary therapy is encapsulated cell therapy (such as that delivering Ciliary Neurotrophic Factor (CNTF)). See, e.g., Sieving, P. A. et al, 2006. Proc Natl Acad Sci USA, 103(10):3896-3901, which is hereby incorporated by reference. In another embodiment, the secondary therapy is a neurotrophic factor therapy (such as pigment epithelium-derived factor, PEDF; ciliary neurotrophic factor 3; rod-derived cone viability factor (RdCVF) or glial-derived neurotrophic factor). In another embodiment, the secondary therapy is anti-apoptosis therapy (such as that delivering X-linked inhibitor of apoptosis, XIAP). In yet another embodiment, the secondary therapy is rod-derived cone viability factor 2. The secondary therapy can be administered before, concurrent with, or after administration of any of the CFI polypeptides or biologically active fragments and/or variants thereof described above.

In some embodiments, any of the CFI polypeptides or biologically active fragments and/or variants thereof disclosed herein is administered to a subject in combination with another therapeutic agent or therapeutic procedure. In some embodiments, the additional therapeutic agent is an anti-VEGF therapeutic agent (e.g., such as an anti-VEGF antibody or fragment thereof such as ranibizumab, bevacizumab or aflibercept), a vitamin or mineral (e.g., vitamin C, vitamin E, lutein, zeaxanthin, zinc or copper), omega-3 fatty acids, and/or Visudyne™. In some embodiments, the other therapeutic procedure is a diet having reduced omega-6 fatty acids, laser surgery, laser photocoagulation, submacular surgery, retinal translocation, and/or photodynamic therapy. In some embodiments, the additional therapeutic agent is a vector (e.g., an AAV vector) encoding a CFI protein or biologically active fragment/variant thereof and/or a CFH protein or a biologically active fragment/variant thereof.

In some embodiments, any of the CFI polypeptides or biologically active fragments and/or variants thereof disclosed herein is administered to a subject in combination with an additional agent needed for processing and/or improving the function of the CFI polypeptides or biologically active fragments and/or variants thereof. For example, the CFI polypeptide or biologically active fragment and/or variant thereof may be administered with an antibody (or a vector encoding that antibody) that potentiates the activity of an endogenous CFH protein. Examples of such antibodies are found in WO2016/028150 or WO2019139481, which are each incorporated herein in their entirety. In some embodiments, the CFI polypeptide or biologically active fragment and/or variant thereof is administered in combination with a processing protein (e.g., a protease) or a vector encoding a processing protein. In some embodiments, the processing protein is a protease (e.g., a furin protease). However, in alternative embodiments, any of the CFI polypeptides or biologically active fragments and/or variants thereof disclosed herein is not administered with a processing polypeptide (or a vector encoding that processing polypeptide). For example, in some embodiments, the disclosure contemplates methods of administering a CFI polypeptide or biologically active fragment and/or variants thereof, wherein the CFI polypeptide or biologically active fragment and/or variant thereof is not administered in combination with a processing polypeptide (e.g., a furin) or a vector encoding a processing polypeptide (e.g., a furin). In some embodiments, the disclosure contemplates a composition comprising any of the CFI polypeptides or biologically active fragments and/or variants thereof disclosed herein, wherein that composition does not include any additional processing polypeptide (e.g., furin) or vector encoding a processing polypeptide (e.g., furin). In some embodiments, the disclosure contemplates administering a CFI polypeptide or biologically active fragment and/or variant thereof to a patient, wherein the patient utilizes endogenous sources of a processing polypeptide (e.g., furin) to process the CFI protein to its mature form. That is, in some embodiments, the compositions disclosed herein are capable of being processed to active CFI upon administration of the CFI polypeptide or biologically active fragment and/or variant thereof to the patient. In some embodiments, the compositions of the present disclosure, used according to the methods disclosed herein, are capable of being processed to active CFI.

### Kits

In some embodiments, any of the CFI polypeptides or biologically active fragments and/or variants thereof disclosed herein is assembled into a pharmaceutical or diagnostic or research kit to facilitate their use in therapeutic, diagnostic or research applications. A kit may include one or more containers housing any of the CFI polypeptides or biologically active fragments and/or variants thereof disclosed herein and instructions for use. In some embodiments, the kit includes instructions for administering any of the CFI polypeptides or biologically active fragments and/or variants thereof to a subject in whom it has been determined has one or more of any of the CFI mutations disclosed herein. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit.

### Examples

The disclosure now being generally described, it will be more readily understood by reference to the following examples, which are included merely for purposes of illustration of certain embodiments and embodiments of the present disclosure, and are not intended to limit the disclosure.

### Example 1: CFI Mutant Analysis

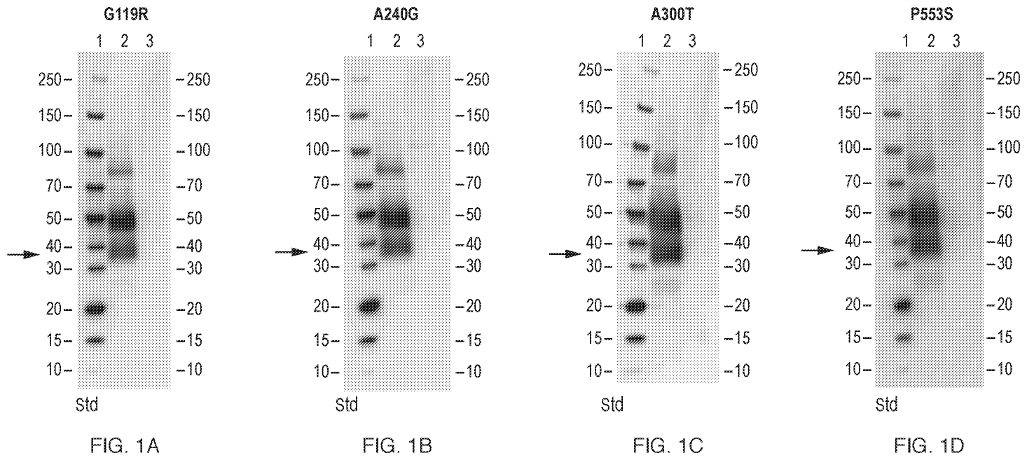

This example was designed to assess the impact of certain CFI mutations on the activity of CFI. Several CFI mutant variants were produced and characterized in a functional assay. Specifically, G119R, A240G, P553S, and A300T variants were expressed in cells that were co-transfected with a gene encoding furin, and the expressed CFI protein was purified using an affinity column. As shown in FIGS. 1A-1D, mature mutant CFI proteins were produced.

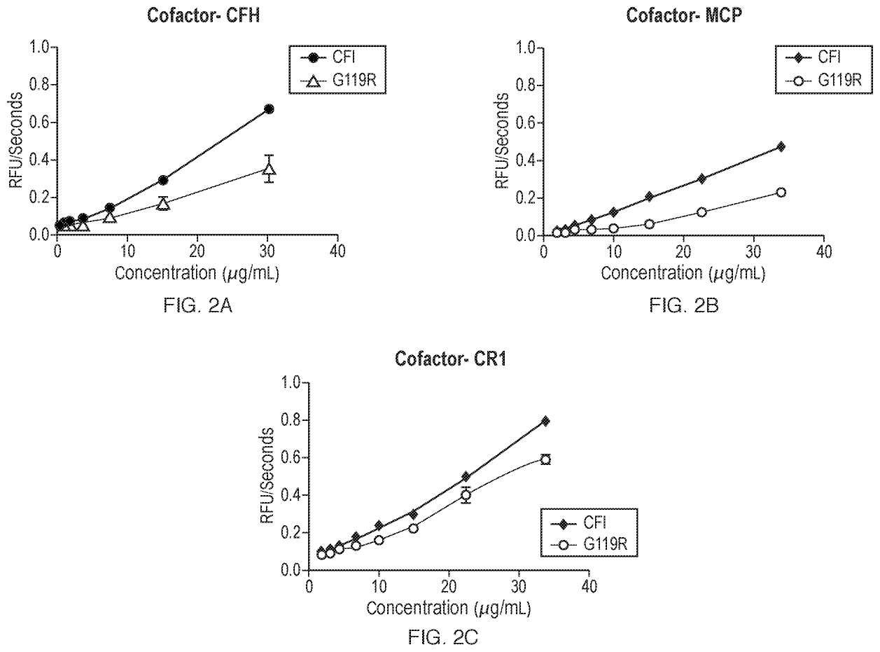

This example shows that CFI mutant variants can have an impact on CFI function. Activity of the G119R, A240G, P553S, and A300T mutant CFI proteins was tested in a fluorescence cofactor assay. Experimental details are provided below in the section (CFI Activity Assay Protocol). Briefly, C3b was labeled with ANS, which provides a fluorescent signal. The ANS-labeled C3b was then mixed with one of three different cofactors: CFH, CR1 or MCP. These cofactors bind to CFH-, CR1- or MCP-binding domains of C3b. Increasing concentrations of CFI variants (G119R, A240G, P553S, or A300T) or wild type CFI was then added to each cofactor/ANS-C3b mixture to initiate cleavage of C3b to iC3b. Cleavage of C3b was reflected by the change in relative fluorescent units (RFUs) over time. FIGS. 2A-2C (G119R CFI protein), 3A-3C (A240G CFI protein), 4A-4C (P553S CFI protein), and 5A-5C (A300T CFI protein) show the results from the fluorescence cofactor assays.

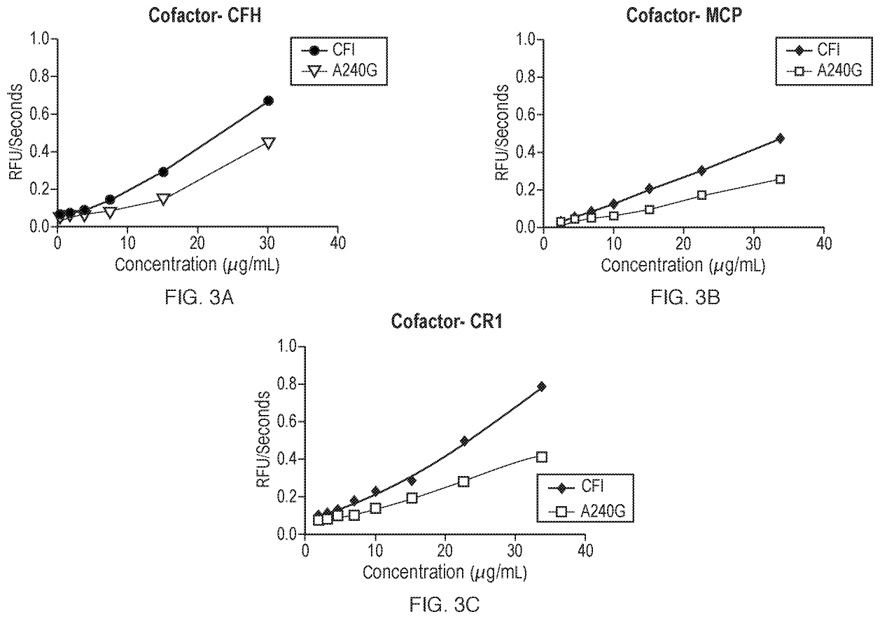

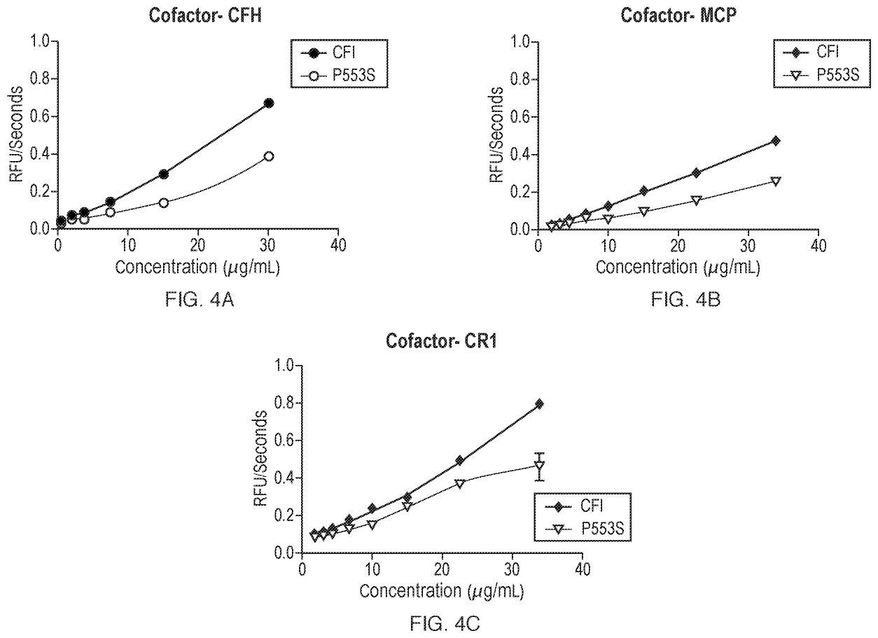

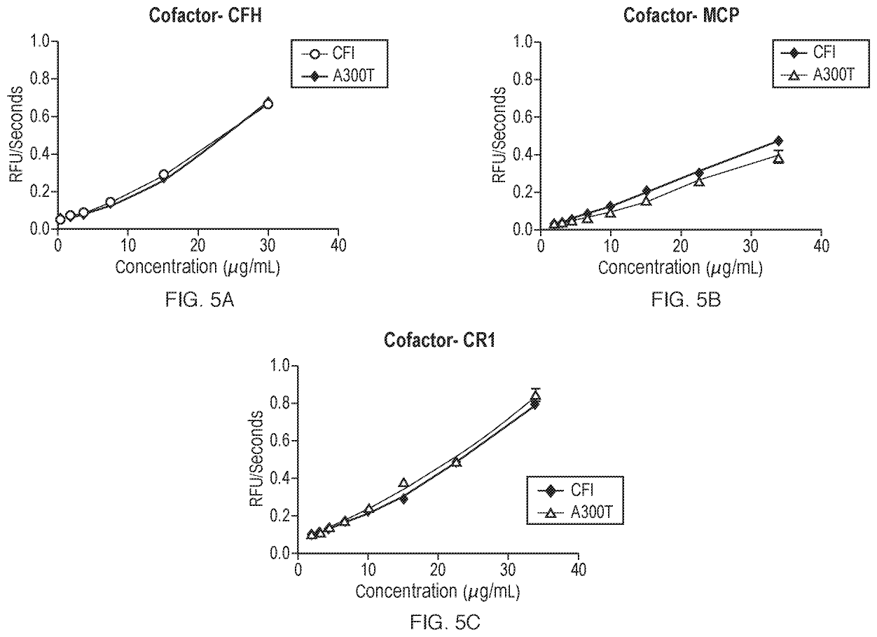

Surprisingly, it was found that certain CFI mutations (G119R, A240G, and P553S) were associated with greatly reduced CFI protein function (FIGS. 2A-2C, 3A-3C, and 4A-4C). By comparison, the A300T mutation appeared to have relatively little impact on CFI function (FIGS. 5A-5C). These data suggest that patients (e.g., an AMD patient) harboring mutations (e.g., G119R, A240G, or P553S) that reduce CFI activity may be more amenable to treatment with any of the CFI polypeptides or biologically active fragments and/or variants thereof disclosed herein than, for example, a patient (e.g., an AMD patient) lacking these mutations or a patient having a CFI mutation that does not have a significant impact on CFI activity (e.g., A300T).

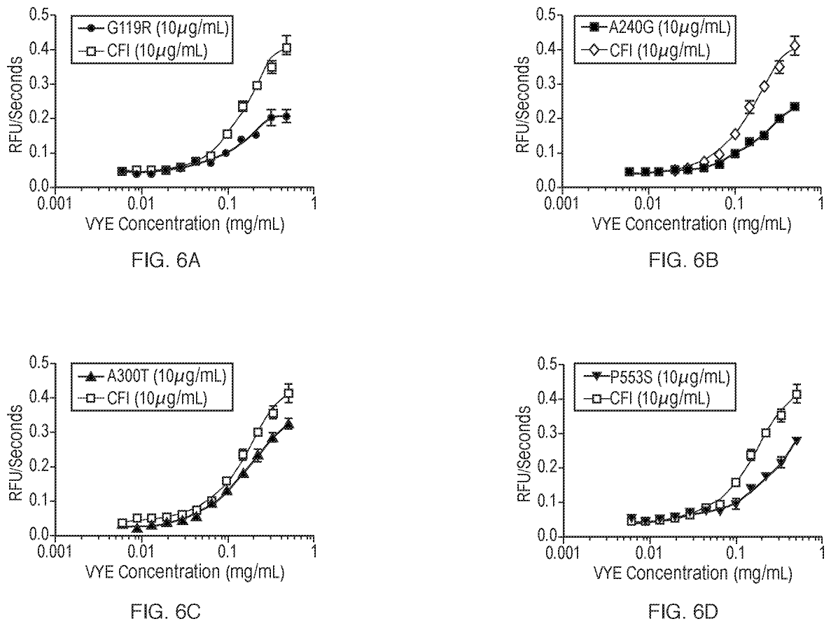

In a separate experiment, increasing concentrations of CFH were added to a mixture of a fixed concentration of ANS-C3b and wild type CFI or a CFI variant (G119R, A240G, P553S, and A300T). Similar results were obtained from this experiment (FIGS. 6A-6D).

### CFI Activity Assay Protocol

Concentrated stock of ANS (ARCOS Organics #401210051) was prepared by weighing out ANS into 1 ml DMSO in a glass amber vial. 1 mL of ANS working stock (500 μM) was prepared by diluting 0.5 μl of concentrated stock with 1xTBS in a polypropylene “Eppendorf” type tube and stored at room temperature until use. 1 mL of dilute CFH (Complement Technology, Inc. Cat #A137) was prepared for each 96 well plate. CompTech plasma derived CFH material was diluted 1:5 in 1xTBS from 1.0 mg/mL to 0.2 mg/ml. The materials were then stored in an ice water bath until use. CFI standard curve samples were prepared in 1xTBS in duplicate. The test/unknown samples were diluted as appropriate in 1xTBS and then stored in ice water bath until use. The standard controls included “no C3b,” “no CFI,” and “no CFH”. The plate reader was warmed to 30° C., and the 96 well plate was placed on ice or cold pack. 20 μL of C3b (Complement Technology, Cat #A114) was then plated at 1 mg/mL per well (except no C3b control wells), and 10 μL of ANS working stock per well was added. 10 μL of CFH was added to appropriate wells. The well contents were mixed briefly (less than 1 min) on a plate shaker at 4000 rpm. The plate was placed in a plate reader to warm to 30° C. The plate was removed and 10 μL of CFI standards (wild type CFI protein) or samples (CFI mutant variants) were added per well (except no CFI was added for control wells). The plate was read for 30 minutes every fifteen seconds at 30° C. on a SpectraMax M3 plate reader in kinetic mode with excitation set at 386 nm and emission set at 472 nm. Reactions were stopped by adding reducing Laemmli buffer and run on a gel to visualize C3b cleavage using Coomasie stain. The slope of kinetic reaction (measured between 300 and 900 seconds) was plotted versus concentration of FI standard curve and unknowns were interpolated.

### NUMBERED EMBODIMENTS

Embodiments disclosed herein include embodiments P1 to P74 as provided in the numbered embodiments of the disclosure.

Embodiment P1: A method of treating a subject having a disease or disorder associated with undesired activity of the alternative complement pathway, comprising the step of administering to the subject a complement factor I (CFI) protein or biologically active fragment and/or variant thereof, wherein the subject has one or more CFI gene mutations.

Embodiment P2: A method of treating a subject having age-related macular degeneration (AMD), comprising the step of administering to the subject a complement factor I (CFI) protein or biologically active fragment and/or variant thereof, wherein the subject has one or more CFI gene mutations.

Embodiment P3: The method of embodiment P1 or P2, wherein the CFI protein or biologically active fragment and/or variant thereof comprises a heavy chain and a light chain.

Embodiment P4: The method of any one of embodiments P1-P3, wherein the CFI protein or biologically active fragment and/or variant thereof comprises a FIMAC domain.

Embodiment P5: The method of any one of embodiments P1-P4, wherein the CFI protein or biologically active fragment and/or variant thereof comprises a Scavenger Receptor Cysteine Rich (SRCR) domain.

Embodiment P6: The method of any one of embodiments P1-P5, wherein the CFI protein or biologically active fragment and/or variant thereof comprises at least one LDL receptor Class A domain.

Embodiment P7: The method of any one of embodiments P1-P6, wherein the CFI protein or biologically active fragment and/or variant thereof comprises two LDL receptor Class A domains.

Embodiment P8: The method of any one of embodiments P1-P7, wherein the CFI protein or biologically active fragment and/or variant thereof comprises a serine protease domain.

Embodiment P9: The method of any one of embodiments P1-P8, wherein the CFI protein or biologically active fragment and/or variant thereof comprises a FIMAC domain, a Scavenger Receptor Cysteine Rich (SRCR) domain, and two LDL receptor Class A domains.

Embodiment P10: The method of any one of embodiments P1-P9, wherein the CFI protein or biologically active fragment and/or variant thereof is capable of cleaving C3b and C4b proteins.

Embodiment P11: The method of any one of embodiments P1-P10, wherein the CFI protein or biologically active fragment and/or variant thereof is capable of inhibiting the assembly of C3 and C5 convertase enzymes.

Embodiment P12: The method of any one of embodiments P1-P11, wherein the CFI protein or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 1, or a biologically active fragment thereof.

Embodiment P13: The method of any one of embodiments P1-P11, wherein the CFI protein or biologically active fragment and/or variant thereof comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 2, or a biologically active fragment thereof.

Embodiment P14: The method of any one of embodiments P1-P13, wherein the CFI protein or biologically active fragment and/or variant thereof is encoded by a nucleotide sequence that is at least 70%, 75%, 80%, 85%, 90%, 91%, 92%, 93%, 94%, 95%, 96%, 97%, 98%, 99% or 100% identical to the nucleotide sequence of any one of SEQ ID NOs: 3-6, or a biologically active fragment thereof.

Embodiment P15: The method of any one of embodiments P1-P14, wherein the CFI protein or biologically active fragment and/or variant thereof is a mature CFI protein or biologically active fragment and/or variant thereof.

Embodiment P16: The method of any of embodiments P1-P15, wherein the subject is not administered a protease or a polynucleotide encoding a protease.

Embodiment P17: The method of any of embodiments P1-P16, wherein the subject is not administered a furin protease or a polynucleotide encoding a furin protease.

Embodiment P18: The method of any one of embodiments P1-P17, wherein the subject is a human.

Embodiment P19: The method of embodiment P18, wherein the human is at least 40 years of age.

Embodiment P20: The method of embodiment P18, wherein the human is at least 50 years of age.

Embodiment P21: The method of embodiment P18, wherein the human is at least 65 years of age.

Embodiment P22: The method of any one of embodiments P1-P21, wherein the CFI protein or biologically active fragment and/or variant thereof is administered locally.

Embodiment P23: The method of any one of embodiments P1-P21, wherein the CFI protein or biologically active fragment and/or variant thereof is administered systemically.

Embodiment P24: The method of any one of embodiments P1-P23, wherein the subject has a loss-of-function mutation in the subject's CFI gene.

Embodiment P25: The method of any one of embodiments P1-P24, wherein the subject has one or more CFI mutations selected from the group consisting of: G119R, L131R, V152M, G162D, R187Y, R187T, T2031, A240G, A258T, G287R, A300T, R317W, R339Q, V412M, and P553S.

Embodiment P26: The method of any one of embodiments P1-P25, wherein the subject has one or more CFI mutations selected from the group consisting of: P553S, K441R, R339Q, R339Ter, R317Q, R317W, A300T, G287R, G261D, A258T, A240G, T2031, R187Q, R187Ter, G162D, V152M, or G119R.

Embodiment P27: The method of any one of embodiments P1-P24, wherein the subject has a P553S CFI mutation.

Embodiment P28: The method of any one of embodiments P1-P24, wherein the subject has a K441R CFI mutation.

Embodiment P29: The method of any one of embodiments P1-P24, wherein the subject has an R339Q CFI mutation.

Embodiment P30: The method of any one of embodiments P1-P24, wherein the subject has an R339Ter CFI mutation.

Embodiment P31: The method of any one of embodiments P1-P24, wherein the subject has an R317Q CFI mutation.

Embodiment P32: The method of any one of embodiments P1-P24, wherein the subject has an R317W CFI mutation.

Embodiment P33: The method of any one of embodiments P1-P24, wherein the subject has an A300T CFI mutation.

Embodiment P34: The method of any one of embodiments P1-P24, wherein the subject has a G287R CFI mutation.

Embodiment P35: The method of any one of embodiments P1-P24, wherein the subject has a G261D CFI mutation.

Embodiment P36: The method of any one of embodiments P1-P24, wherein the subject has an A258T CFI mutation.

Embodiment P37: The method of any one of embodiments P1-P24, wherein the subject has an A240G CFI mutation.

Embodiment P38: The method of any one of embodiments P1-P24, wherein the subject has a T2031 CFI mutation.

Embodiment P39: The method of any one of embodiments P1-P24, wherein the subject has an R187Q CFI mutation.

Embodiment P40: The method of any one of embodiments P1-P24, wherein the subject has an R187Ter CFI mutation.

Embodiment P41: The method of any one of embodiments P1-P24, wherein the subject has a G162D CFI mutation.

Embodiment P42: The method of any one of embodiments P1-P24, wherein the subject has a V152M CFI mutation.

Embodiment P43: The method of any one of embodiments P1-P24, wherein the subject has a G119R CFI mutation.

Embodiment P44: The method of any one of embodiments 1-P43, wherein the subject is homozygous for at least one of the one or more CFI mutations.

Embodiment P45: The method of any one of embodiments P1-P44, wherein the subject is heterozygous for at least one of the one or more CFI mutations.

Embodiment P46: The method of any one of embodiments P1-P45, wherein the subject expresses a mutant CFI protein having reduced CFI activity as compared to a wildtype CFI protein (e.g., a CFI protein having the amino acid sequence of SEQ ID NO: 1).

Embodiment P47: The method of embodiment P46, wherein the CFI activity is the ability to cleave C3b to iC3b.

Embodiment P48: The method of any one of embodiments P1-P47, wherein if a CFI protein having the CFI mutation were tested in a functional assay, the mutant CFI protein would display reduced CFI activity as compared to a wildtype CFI protein (e.g., a CFI protein having the amino acid sequence of SEQ ID NO: 1).

Embodiment P49: The method of embodiment P48, wherein the functional assay tests the ability of CFI to cleave C3b to iC3b.

Embodiment P50: The method of any one of embodiments P1-P49, wherein the subject has atypical hemolytic uremic syndrome (aHUS).

Embodiment P51: The method of any one of embodiments P1-P50, wherein the subject is suffering from a renal disease or complication.

Embodiment P52: The method of any one of embodiments P1-P51, wherein the subject is a subject in whom it has been determined has one or more CFI mutations.

Embodiment P53: The method of embodiment P52, wherein the subject is a subject in whom it has been determined has one or more CFI mutations selected from the group consisting of: G119R, L131R, V152M, G162D, R187Y, R187T, T2031, A240G, A258T, G287R, A300T, R317W, R339Q, V412M, and P553S.

Embodiment P54: The method of embodiment P52, wherein the subject is a subject in whom it has been determined has one or more CFI mutations selected from the group consisting of: P553S, K441R, R339Q, R339Ter, R317Q, R317W, A300T, G287R, G261D, A258T, A240G, T2031, R187Q, R187Ter, G162D, V152M, or G119R.

Embodiment P55: The method of embodiment P52, wherein the subject is a subject in whom it has been determined has a P553S CFI mutation.

Embodiment P56: The method of embodiment P52, wherein the subject is a subject in whom it has been determined has a K441R CFI mutation.

Embodiment P57: The method of embodiment P52, wherein the subject is a subject in whom it has been determined has an R339Q CFI mutation.

Embodiment P58: The method of embodiment P52, wherein the subject is a subject in whom it has been determined has an R339Ter CFI mutation.

Embodiment P59: The method of embodiment P52, wherein the subject is a subject in whom it has been determined has an R317Q CFI mutation.

Embodiment P60: The method of embodiment P52, wherein the subject is a subject in whom it has been determined has an R317W CFI mutation.

Embodiment P61: The method of embodiment P52, wherein the subject is a subject in whom it has been determined has an A300T CFI mutation.

Embodiment P62: The method of embodiment P52, wherein the subject is a subject in whom it has been determined has a G287R CFI mutation.

Embodiment P63: The method of embodiment P52, wherein the subject is a subject in whom it has been determined has a G261D CFI mutation.

Embodiment P64: The method of embodiment P52, wherein the subject is a subject in whom it has been determined has an A258T CFI mutation.

Embodiment P65: The method of embodiment P52, wherein the subject is a subject in whom it has been determined has an A240G CFI mutation.

Embodiment P66: The method of embodiment P52, wherein the subject is a subject in whom it has been determined has a T2031 CFI mutation.

Embodiment P67: The method of embodiment P52, wherein the subject is a subject in whom it has been determined has an R187Q CFI mutation.

Embodiment P68: The method of embodiment P52, wherein the subject is a subject in whom it has been determined has an R187Ter CFI mutation.

Embodiment P69: The method of embodiment P52, wherein the subject is a subject in whom it has been determined has a G162D CFI mutation.

Embodiment P70: The method of embodiment P52, wherein the subject is a subject in whom it has been determined has a V152M CFI mutation.

Embodiment P71: The method of embodiment P52, wherein the subject is a subject in whom it has been determined has a G119R CFI mutation.

Embodiment P72: The method of any one of embodiments P52-P71, wherein the subject is a subject in whom it has been determined is homozygous for at least one of the one or more CFI mutations.

Embodiment P73: The method of any one of embodiments P52-P72, wherein the subject is a subject in whom it has been determined is heterozygous for at least one of the one or more CFI mutations.

Embodiment P74: The method of any one of embodiments P1-P73, wherein the CFI polypeptide or biologically active fragment and/or variant thereof is administered intravitreally.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the disclosure should be determined by reference to the claims, along with their full scope of equivalents, and the specification, along with such variations.

1. A method of treating a subject having a disease or disorder associated with undesired activity of the alternative complement pathway, comprising the step of administering to the subject a complement factor I (CFI) protein or biologically active fragment and/or variant thereof, wherein the subject has one or more CFI gene mutations.

2. A method of treating a subject having age-related macular degeneration (AMD), comprising the step of administering to the subject a complement factor I (CFI) protein or biologically active fragment and/or variant thereof, wherein the subject has one or more CFI gene mutations.

3. The method of claim 1 or 2, wherein the CFI protein or biologically active fragment and/or variant thereof comprises a heavy chain and a light chain.

4. The method of any one of claims 1-3, wherein the CFI protein or biologically active fragment and/or variant thereof comprises a FIMAC domain.

5. The method of any one of claims 1-4, wherein the CFI protein or biologically active fragment and/or variant thereof comprises a Scavenger Receptor Cysteine Rich (SRCR) domain.

6. The method of any one of claims 1-5, wherein the CFI protein or biologically active fragment and/or variant thereof comprises at least one low-density lipoprotein (LDL) receptor Class A domain.

7. The method of any one of claims 1-6, wherein the CFI protein or biologically active fragment and/or variant thereof comprises two LDL receptor Class A domains.

8. The method of any one of claims 1-7, wherein the CFI protein or biologically active fragment and/or variant thereof comprises a serine protease domain.

9. The method of any one of claims 1-8, wherein the CFI protein or biologically active fragment and/or variant thereof comprises a FIMAC domain, a Scavenger Receptor Cysteine Rich (SRCR) domain, and two LDL receptor Class A domains.

10. The method of any one of claims 1-9, wherein the CFI protein or biologically active fragment and/or variant thereof is capable of cleaving C3b and C4b proteins.

11. The method of any one of claims 1-10, wherein the CFI protein or biologically active fragment and/or variant thereof is capable of inhibiting the assembly of C3 and C5 convertase enzymes.

12. The method of any one of claims 1-11, wherein the CFI protein or biologically active fragment and/or variant thereof comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 1, or a biologically active fragment thereof.

13. The method of any one of claims 1-11, wherein the CFI protein or biologically active fragment and/or variant thereof comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the amino acid sequence of SEQ ID NO: 2, or a biologically active fragment thereof.

14. The method of any one of claims 1-13, wherein the CFI protein or biologically active fragment and/or variant thereof is encoded by a nucleotide sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleotide sequence of any one of SEQ ID NOs: 3-6 or a fragment thereof.

15. The method of any one of claims 1-14, wherein the CFI protein or biologically active fragment and/or variant thereof is a mature CFI protein or biologically active fragment and/or variant thereof.

16. The method of any of claims 1-15, wherein the subject is not administered a protease or a polynucleotide encoding a protease.

17. The method of any of claims 1-16, wherein the subject is not administered a furin protease or a polynucleotide encoding a furin protease.

18. The method of any one of claims 1-17, wherein the subject is a human.

19. The method of claim 18, wherein the human is at least 40 years of age.

20. The method of claim 18, wherein the human is at least 50 years of age.

21. The method of claim 18, wherein the human is at least 65 years of age.

22. The method of any one of claims 1-21, wherein the CFI protein or biologically active fragment and/or variant thereof is administered locally.

23. The method of any one of claims 1-21, wherein the CFI protein or biologically active fragment and/or variant thereof is administered systemically.

24. The method of any one of claims 1-23, wherein the subject has a loss-of-function mutation in the subject's CFI gene.

25. The method of any one of claims 1-24, wherein the subject has one or more CFI mutations selected from the group consisting of: G119R, L131R, V152M, G162D, R187Y, R187T, T2031, A240G, A258T, G287R, A300T, R317W, R339Q, V412M, and P553S.

26. The method of any one of claims 1-25, wherein the subject has one or more CFI mutations selected from the group consisting of: P553S, K441R, R339Q, R339Ter, R317Q, R317W, A300T, G287R, G261D, A258T, A240G, T2031, R187Q, R187Ter, G162D, V152M, and G119R.

27. The method of any one of claims 1-24, wherein the subject has a G119R CFI mutation.

28. The method of any one of claims 1-24, wherein the subject has a P553S CFI mutation.

29. The method of any one of claims 1-24, wherein the subject has a K441R CFI mutation.

30. The method of any one of claims 1-24, wherein the subject has an R339Q CFI mutation.

31. The method of any one of claims 1-24, wherein the subject has an R339Ter CFI mutation.

32. The method of any one of claims 1-24, wherein the subject has an R317Q CFI mutation.

33. The method of any one of claims 1-24, wherein the subject has an R317W CFI mutation.

34. The method of any one of claims 1-24, wherein the subject has an A300T CFI mutation.

35. The method of any one of claims 1-24, wherein the subject has a G287R CFI mutation.

36. The method of any one of claims 1-24, wherein the subject has a G261D CFI mutation.

37. The method of any one of claims 1-24, wherein the subject has an A258T CFI mutation.

38. The method of any one of claims 1-24, wherein the subject has an A240G CFI mutation.

39. The method of any one of claims 1-24, wherein the subject has a T2031 CFI mutation.

40. The method of any one of claims 1-24, wherein the subject has an R187Q CFI mutation.

41. The method of any one of claims 1-24, wherein the subject has an R187Ter CFI mutation.

42. The method of any one of claims 1-24, wherein the subject has a G162D CFI mutation.

43. The method of any one of claims 1-24, wherein the subject has a V152M CFI mutation.

44. The method of any one of claims 1-43, wherein the subject is homozygous for at least one of the one or more CFI mutations.

45. The method of any one of claims 1-44, wherein the subject is heterozygous for at least one of the one or more CFI mutations.

46. The method of any one of claims 1-45, wherein the one or more CFI mutations reduce CFI activity as compared to a wild type CFI protein.

47. The method of claim 46, wherein the CFI activity is the ability to cleave C3b to iC3b.

48. The method of claim 46, wherein the wild type CFI protein comprises the amino acid sequence of SEQ ID NO: 1.

49. The method of any one of claims 1-48, wherein the subject has atypical hemolytic uremic syndrome (aHUS).

50. The method of any one of claims 1-49, wherein the subject is suffering from a renal disease or complication.

51. The method of any one of claims 1-50, wherein the subject has been determined to have the one or more CFI mutations.

52. The method of claim 51, wherein the one or more CFI mutations are selected from the group consisting of: G119R, L131R, V152M, G162D, R187Y, R187T, T2031, A240G, A258T, G287R, A300T, R317W, R339Q, V412M, and P553S.

53. The method of claim 51, wherein the one or more CFI mutations are selected from the group consisting of: P553S, K441R, R339Q, R339Ter, R317Q, R317W, A300T, G287R, G261D, A258T, A240G, T2031, R187Q, R187Ter, G162D, V152M, and G119R.

54. The method of any one of claims 1-53, wherein the CFI polypeptide or biologically active fragment and/or variant thereof is administered intravitreally.
